(19) United States
(12) Patent Application Publication
(10) Pub. No.: US 2013/0274719 A1
(43) Pub. Date: Oct. 17, 2013

(54) CUTANEOUS ABSCESS DRAINAGE DEVICE AND METHODS

(71) Applicants: Richard E. Berkey, Portland, OR (US); Aloysius N. Fobi, Gresham, OR (US)

(72) Inventors: Richard E. Berkey, Portland, OR (US); Aloysius N. Fobi, Gresham, OR (US)

(21) Appl. No.: 13/829,466

(22) Filed: Mar. 14, 2013

Related U.S. Application Data
(60) Provisional application No. 61/622,635, filed on Apr. 11, 2012.

(51) Int. Cl. A61B 17/34

(52) U.S. Cl. CPC A61B 17/3415
USPC 604/543

(57) ABSTRACT

An abscess draining device includes a puncture member and a drain member. The puncture member includes a distal end configured to advance into and out of an abscess to form spaced apart first and second incisions. The drain member is connected to a proximal end of the puncture member. Advancing the puncture member through the abscess positions the drain member extending through the first incision, the abscess, and the second incision. The drain member is disconnectable from the puncture member after being positioned extending through the first incision, the abscess, and the second incision.

### CROSS-REFERENCE TO RELATED APPLICATION

This application claims the benefit of the filing date of U.S. Provisional Application No. 61/622,635, filed Apr. 11, 2012, and entitled CUTANEOUS ABSCESS DRAINAGE DEVICE AND METHODS, the disclosure of which is incorporated, in its entirety, by reference.

### TECHNICAL FIELD

The present application related to treating infections, and more particularly relates to systems and methods for treating cutaneous abscesses.

### BACKGROUND

Cutaneous abscesses are localized infections characterized by a collection or pus. Although proper treatment requires drainage of the fluid collection, the health care provider's approach depends on certain factors such as size and location of the abscess. Because the pain associated with infiltrating a local anesthetic can be significant, the patient's ability to tolerate the injections required to achieve adequate anesthesia before incision and drainage may also affect the approach. At times, patients even decline or defer appropriate care due to fear or pain associated with injecting enough local anesthetic to adequately numb the area.

Treatment with incision and drainage has historically been performed by cleaning the skin over the abscess, injecting a local anesthetic, incising the skin over the abscess with a scalpel to cut through the skin into the abscess cavity, expressing pus from the wound, using a hemostat to explore the wound and to break up pockets or loculations, and inserting packing material (frequently a strip of gauze) to allow continued drainage from the opening (which prevents skin closure and re-accumulation of the abscess). Follow-up for packing changes and removal is required. FIGS. 7A and 7B illustrate a tissue layer 22 that is cut with a scalpel 18 to form an incision 24 that provides access to an abscess cavity 20. The abscess cavity 20 is drained and a packing member 29 is inserted through the incision 24 into the cavity 20. Inserting and removing the packing 29 can be painful, and generally requires a repeat visit.

FIGS. 6A-6E illustrate an alternative method that uses a sterile silicon vessel loop as a drain member 14. The drain member 14 is passed through two separate small incisions 24, 26 made with a scalpel 18 at different locations on a skin surface 22 to provide access to an abscess cavity 20. The drain member 14 is advanced through the incisions 24, 26 and the abscess cavity 20 with a grasping device 16, such as a hemostat. The grasping device 16 includes handles 60 and a grasping portion 62. Ends 50, 52 of the drain member 14 are then tied loosely in a knot 54 over the skin surface 22 to form a loop structure. The drain member 14 is used to help drain fluids 28 from the abscess cavity 20. The drain member 14 remains in place until the abscess heals, thereby eliminating the need for changing and removal of packing that is used in the example of FIGS. 7A-7B.

While the vessel loop technique of FIGS. 6A-6E offers some advantages over standard incision and drainage with packing method of FIGS. 7A-7B, the medical community would welcome additional instruments or techniques for abscess drainage that are easier to perform, provide more consistent results, and provide improved patient comfort.

### SUMMARY

One aspect of the present disclosure is directed to a method of treating a cutaneous abscess that includes providing a puncture member having a drain member attached to a proximal end of thereof, inserting the puncture member through a skin surface and into the abscess, the puncture member forming a first incision, advancing the puncture member from the abscess and through the skin surface to form a second incision, the drain member extending through the second incision, the abscess, and the first incision, detaching the drain member from the puncture member, and leaving the first and second incisions open to promote drainage of fluids from the abscess.

The method may also include using a hemostat to insert and advance the puncture member. Detaching the drain member from the puncture member may include breaking off the drain member. Detaching the drain member from the puncture member may include cutting the drain member with a cutting feature of the puncture member. The method may include forming a loop with the drain member after detaching the drain member from the puncture member. Forming the loop may include tying opposing ends of the drain member together to form a knot. The puncture member may include a tapered construction, and advancing the puncture member through the first and second incisions dilates the first and second incisions. Opening the first and second incisions with the drain member may include pulling the drain member away from the abscess. The method may include dilating the first and second incisions with the puncture member.

Another aspect of the present disclosure relates to a method of draining an abscess. The method includes providing a puncture member, a drain member, and a hemostat, connecting the drain member to a proximal end of the puncture member, grasping the puncture member with the hemostat, inserting the puncture member into the abscess with the hemostat at a first location and exiting the puncture member with the hemostat from the abscess at a second location to position the drain member extending through the abscess, detaching the drain member from the puncture member, and articulating the drain member to drain fluid from the abscess.

The hemostat may include a grasping portion having a shape that matches a shape of an outer surface of the puncture member. The puncture member may form a first incision at the first location and a second incision at the second location. Articulating the drain member may include opening the first and second incisions. The puncture member may include a cutting surface, and detaching the drain member may include contacting the drain member with the cutting surface. The method may include connecting opposing ends of the drain member together outside of the abscess prior to articulating the drain member.

A further aspect of the present disclosure relates to an abscess draining device that includes a puncture member and a drain member. The puncture member includes a distal end configured to advance into and out of an abscess to form spaced apart first and second incisions. The drain member is connected to a proximal end of the puncture member. Advancing the puncture member through the abscess positions the drain member extending through the first incision, the abscess, and the second incision. The drain member is disconnectable from the puncture member after being positioned extending through the first incision, the abscess, and the second incision.

The puncture member may be contoured along its length. The puncture member may be tapered along its length. The puncture member may include a cutting surface configured to sever the drain member from the puncture member. The puncture member may include an increased cross-sectional size from the distal end to the proximal end. The drain member may include a tubular construction. The drain member may include opposed first and second ends that are tied together to form a loop structure after the drain member is positioned extending through the first incision, the abscess, and the second incision. One end of the drain member may be inserted into the proximal end of the puncture member.

Features from any of the above-mentioned embodiments may be used in combination with one another in accordance with the general principles described herein. These and other embodiments, features, and advantages will be more fully understood upon reading the following detailed description in conjunction with the accompanying drawings and claims.

### BRIEF DESCRIPTION OF THE DRAWINGS

The accompanying drawings illustrate a number of exemplary embodiments and are a part of the specification. Together with the following description, these drawings demonstrate and explain various principles of the instant disclosure.

FIGS. 1A-1E show an example abscess draining system treating an abscess in accordance with the present disclosure.

FIGS. 2A-2B show a puncture member and drain member of the abscess draining system of FIGS. 1A-1E in a side and top view.

FIG. 2C shows the puncture member and drain member of FIGS. 2A-2B being handled with a hemostat.

FIGS, 3A-3G show cross sectional shapes for the puncture member of FIGS. 2A-2C.

FIGS. 4A-4C show example cutting features of the puncture member of FIGS. 2A-2C.

FIGS. 5A-5C show example hemostat constructions.

FIGS. 6A-6E show an alternative draining system treating an abscess in accordance with the prior art.

FIGS. 7A-7B show an alternative abscess draining system for treating an abscess in accordance with the prior art.

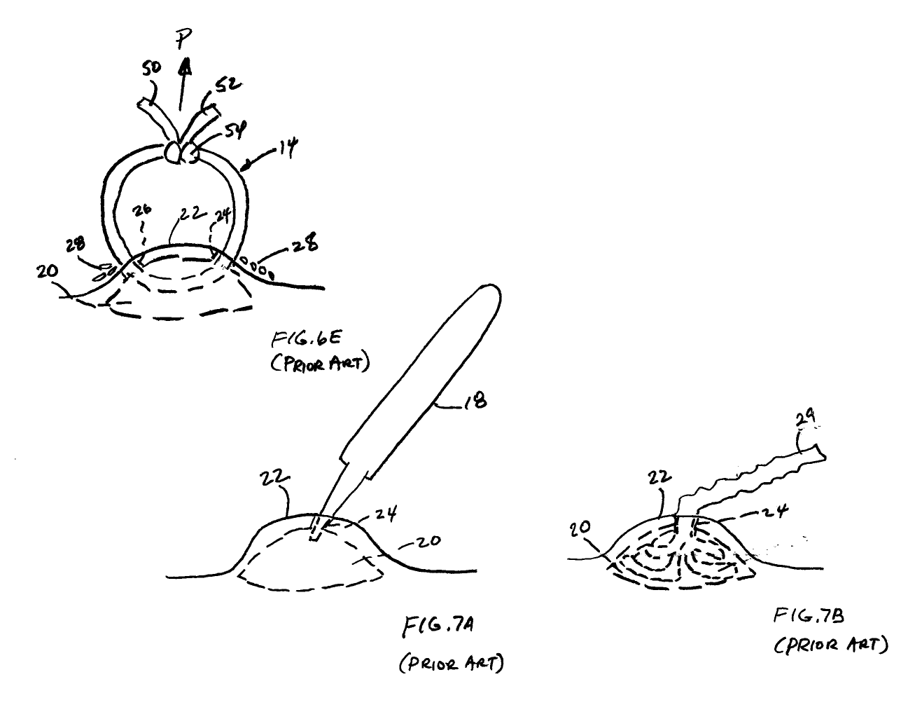

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION OF THE DRAWINGS

The present disclosure is directed to devices, systems and related methods for treating a cutaneous abscess. An example abscess draining device includes a sterile pointed instrument (also referred to as a puncture member) with a flexible drain (also referred to as a drain member) that comprises silicon or other inert material and that is attached to a proximal end or base of the pointed instrument. The pointed instrument is used to puncture the skin over a fluid-filled abscess. The pointed instrument is then advanced (using a hemostat, needle driver or other grasping device) through the abscess cavity, thereby creating a second puncture (exit site). Once the pointed instrument and drain are advanced through the abscess cavity via entrance and exit sites, the drain is detached from the pointed instrument. The drain may be detached by applying traction against a sharp edge within a hollow base of the pointed instrument to detach the drain and leave the drain protruding from the entrance and exit sites. Opposing ends of the drain may be connected together over the skin (e.g., with a tied knot) and used to maintain the wound open.

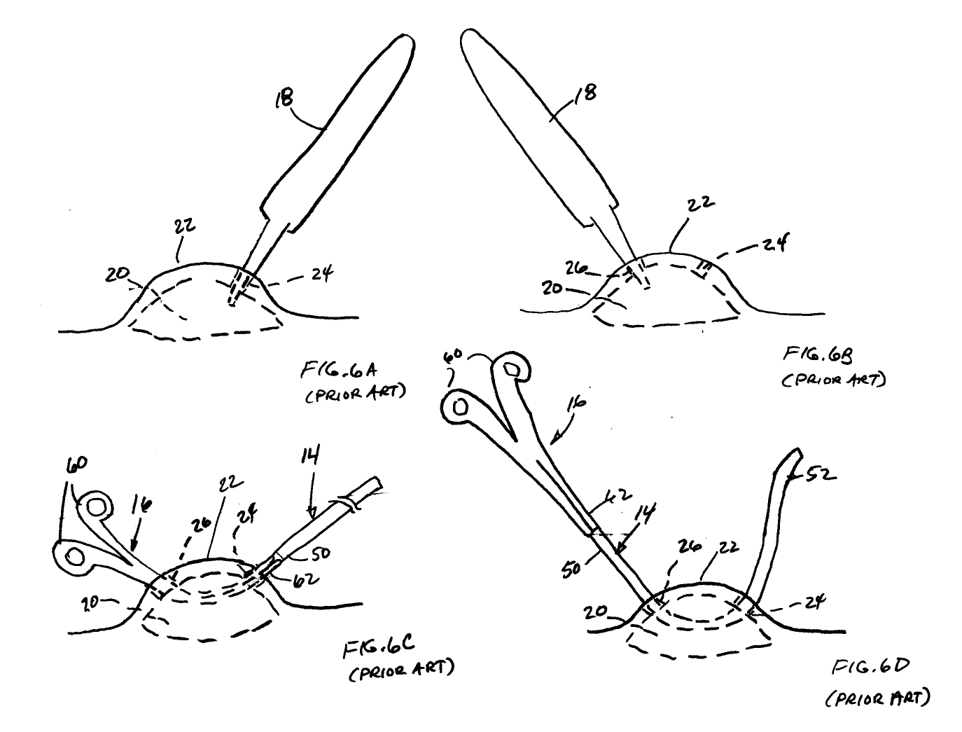

The pointed instrument may be designed to increase in cross-sectional size from distal to proximal ends (e.g., tip to tail) to provide dilating of the entrance and exit puncture sites as the pointed instrument is passed through the abscess. Pus and other fluids may be expressed from the entrance and exit puncture sites by pressing on the adjacent tissue to empty the abscess cavity. A hemostat may be inserted through the entrance and exit puncture sites to break up pockets or loculations in the customary fashion after the drain has been positioned extending through the abscess. After positioning the drain using the pointed instrument, wound care may be similar to that for other vessel loop drainage techniques as described with reference to FIGS. 6A-6E using warm compresses and gentle movement of the drain until healed.

There are numerous potential advantages to using a pointed puncture device rather than making a larger incision or inconsistently sized, shaped and spaced apart incisions, including, for example, increased patient comfort, ease in performing the procedure, decreased bleeding, decreased scarring, and simplified wound care. The pointed instrument of the abscess drainage device may materially differ from a standard suture needle because it not only cuts or punctures skin, but it may also dilate the site to allow an incision big enough for abscess drainage, carry a drain through the wound, and provide a self-contained sharp edge at the base to cut the drain.

The pointed instrument may come in various sizes. The pointed instrument may have various shapes such as being straight or curved. The pointed instrument may have various degrees of concavity or curvature along its length. The pointed instrument may have various types of geometry at the tip. For example, the pointed instrument tip may include a tip size or shape used for standard suture needles (e.g., tapered, cutting, reverse cutting, trocar point or taper cut, side cutting or spatula point, blunt point, or other shapes). The pointed instrument may get wider toward the proximal end in order to dilate the puncture site. The pointed instrument may have various shapes such as round, flat, rectangular, or square, and could contain one or more cutting edges.

The drain attaches to the proximal end of the pointed instrument, or insets into a cavity at the proximal end. The drain may be narrower than the proximal end of the pointed instrument to permit easier passage through the entrance and exit puncture sites in the abscess. The drain may be detached from the device after the drain has passed through both entrance and exit puncture sites. The detachment may occur by applying traction on the drain against a cutting edge at the proximal end the pointed instrument. The detachment may be done with other means such as, for example, providing a perforation or weakened portion in the drain that permits breaking, tearing, or bending that detaches the pointed instrument from the drain.

The pointed instrument and drain may be advanced through the abscess using a standard needle driver, although larger (thicker) sizes or different shaped devices may require a special tool to advance the pointed instrument and drain safely through the abscess.

The advantages of the techniques disclosed herein may include improved cosmetic results with decreased scarring, simplification of dressing changes (i.e., patients are instructed to use warm compresses and to move the knot back and forth multiple times daily), fewer return visits, and potentially reduced costs.

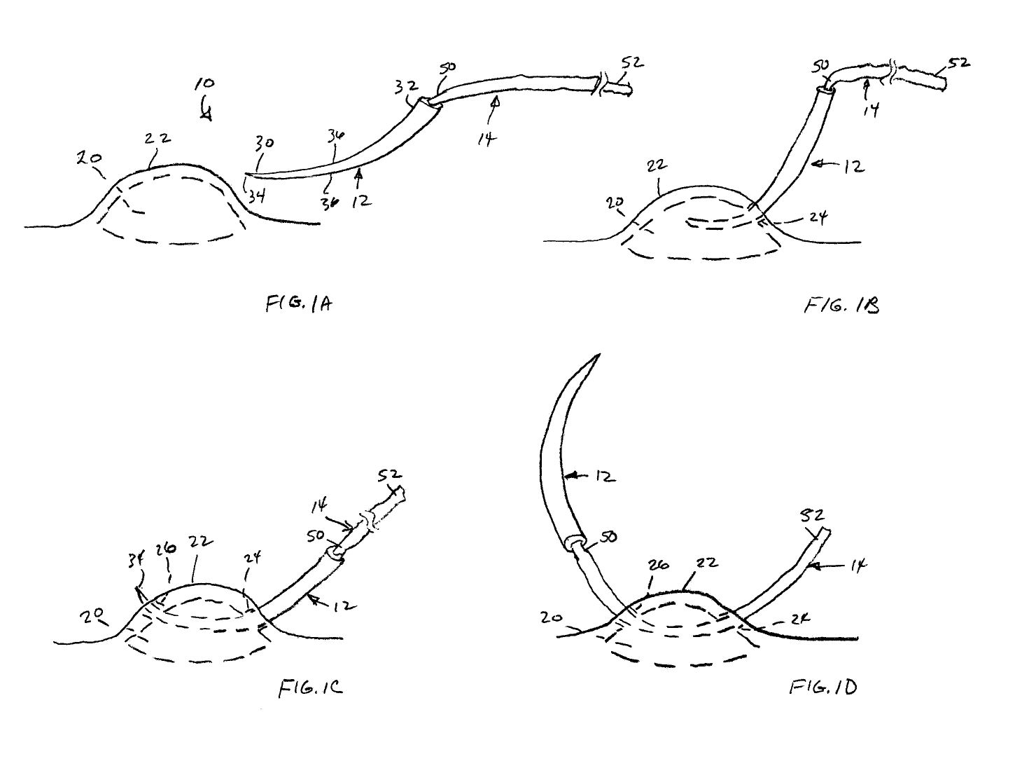

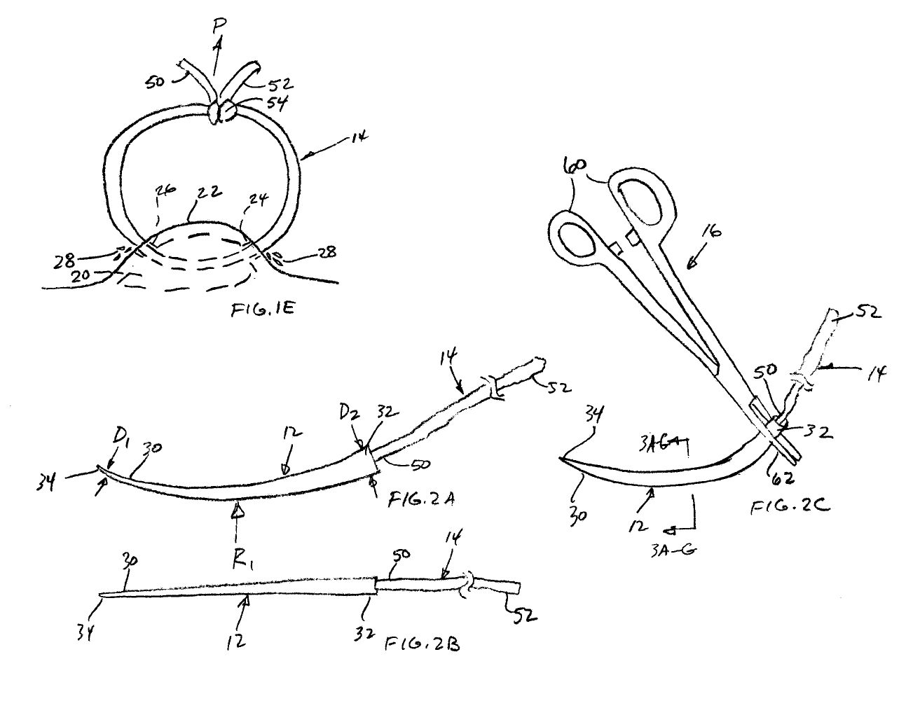

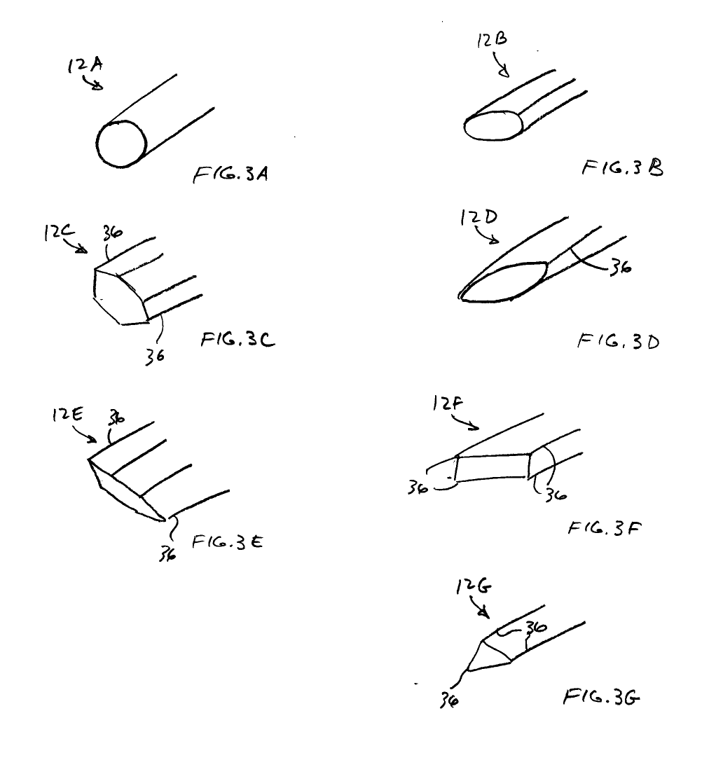

Referring now to FIGS. 1A-1E and FIGS. 2A-2C, an example abscess draining system 10 is shown and described. The abscess draining system 10 includes a puncture member 12 (also referred to as a pointed instrument), a drain member 14 (also referred to as a drain loop or loop member), and a grasping member 16. The grasping member 16 may be optional and used for maneuvering the puncture member 12 relative to an abscess. In some arrangements, the puncture member 12 is of a size and shape that permits easy manipulation by an operator without the use of a grasping member. The drain member 14 is connected to the puncture member 12. The drain member 14 is detachable from the puncture member 12. The puncture member 12 or drain member 14 may include features that promote disconnection of the drain member 14 from the puncture member 12.

The puncture member 12 includes distal and proximal end portions 30, 32, a tip 34 at the distal end portion 30, cutting edges 36 (see FIGS. 3C-3G), and a curvature R₁ (see FIG. 2A). The puncture member 12 may have a tapered construction that provides an increase in width from a tip width D₁ to a tail width D₂ (see FIG. 2A). The puncture member 12 may have curvature along its length in at least one plane (see FIGS. 2A and 2B). The curvature R₁ of puncture member 12 may assist in directing the puncture member 12 into and out of an abscess cavity 20. The tapered construction of puncture member 12 along its length may assist in dilating first and second incisions 24, 26 formed by the puncture member 12 as it is inserted into and directed out of a tissue layer 22 when penetrating the abscess cavity 20.

The puncture member 12 may comprise any desired material such as, for example, a relatively rigid polymeric material or a metal or a metal alloy material. The puncture member 12 may have various sizes and shapes depending on the type and size of abscess being treated. FIGS. 3A-3G illustrate various cross-sectional shapes for the puncture member 12. At least some of the cross-sectional shapes include at least one cutting edge 36 that assists in cutting the tissue layer 22 after being punctured by tip 34. A cross-sectional shape of the puncture member 12 may change along its length (e.g., change from the relatively circular cross-sectional shape of FIG. 3A to the oval shape of FIG. 3B). The puncture members of FIGS. 3A-3G are labeled as puncture members 12A-12G.

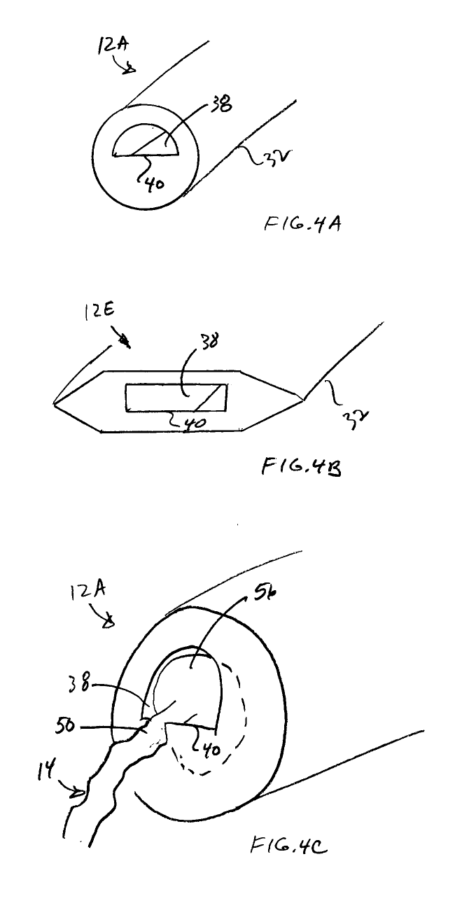

The drain member 14 may be attached to the puncture member 12 by inserting a portion of the drain member 14 into a cavity 38 at the proximal end portion 32 of puncture member 12. FIGS. 4A and 4B show cavity 38 and a drain cutting surface 40 at an entrance into the cavity 38. The drain cutting surface 40 may be configured to cut or at least weaken the drain member 14 to provide detachment of drain member 14 from puncture member 12. FIG. 4C shows a drain member 14 having a distal stop portion 56 inserted into cavity 38 to help retain the drain member 14 connected with the puncture member 12 until the operator intentionally forces the drain member 14 against the drain cutting surface 40, thereby detaching drain member 14 from puncture member 12. Other means for attaching drain member 14 to puncture member 12 may include, for example, using an adhesive that secures the drain member 14 within the cavity 38.

In other embodiments, a coupling structure such as a sleeve, bracket, clip or other fastener may be used to connect the puncture member 12 to drain member 14. In still further embodiments, a portion of the puncture member 12 is inserted into a portion of the drain member 14 such as a bore or aperture that provides temporary connection of puncture member 12 to drain member 14.

The drain member 14 may include distal and proximal ends 50, 52. After detaching the drain member 14 from the puncture member 12, the distal and proximal ends 50, 52 may be connected together using, for example, a knot 54 (see FIG. 1E). When the distal and proximal ends 50, 52 are connected together, the drain member 14 may be arranged in a loop type configuration that permits the operator to grasp the drain member and move the drain member relative to the abscess. FIG. 1E shows application of the force P in a direction perpendicular to the tissue layer 22 and the abscess cavity 20. The drain member 14 may be twisted and moved in any desired direction to manipulate the drain member 14 within the abscess cavity 20. Moving the drain member 14 may also change a flow path for fluids 28 to travel out of the first and second incisions 24, 26 as shown in FIG. 1E. Fluids 28 may also be expressed from the first and second incisions 24, 26 upon puncturing of the tissue layer 22 with the puncture member 12 prior to positioning drain member 14 extending through the first and second incisions 24, 26.

The drain member 14 may comprise any desired material. In at least one example, the drain member 14 comprises a polymeric material and may have flexible properties. The drain member 14 may comprise elastic materials. The drain member 14 may have a tubular construction. Drain member 14 may have any desired cross-sectional shape including, for example, a circular or oval cross-sectional shape.

Referring now to FIGS. 1A-1E, an example method of treating an abscess is described in further detail. FIG. 1A shows the drain member 14 attached to the puncture member 12 and the tip 34 of the puncture member 12 arranged to pierce the tissue layer 22 adjacent to the abscess cavity 20. The puncture member 12 is then inserted through the tissue layer 22 and into the abscess cavity 20 as shown in FIG. 1B to create the first incision 24. The puncture member 12 is further advanced through the abscess cavity 20 and out through the tissue layer 22 at a separate location to form a second incision 26 as shown in FIG. 1C. Advancing the puncture member 12 through the first and second incisions 24, 26 may cut the tissue layer 22 and dilate the first and second incisions 24, 26.

FIG. 1D shows the puncture member 12 advanced completely through the first and second incisions 24, 26 to position the drain member 14 extending through the first and second incisions 24, 26 and the abscess cavity 20. The drain member 14 is then detached from puncture member 12. Any desired method or feature may be used to detach the drain member 14 from the puncture member 12 including, for example, cutting the drain member 14 with a scalpel or other cutting device separate from either the puncture member 12 or the drain member 14. Alternatively, one of the cutting features described with reference to, for example, FIGS. 4A-4C may be used to detach the drain member 14.

The distal and proximal ends 50, 52 of drain member 14 may be connected together. FIG. 1E shows the distal and proximal ends 50, 52 tied together to form a knot 54. A force may be applied to the drain member 14 in a direction P as shown in FIG. 1E. In alternative arrangements, the distal and proximal ends 50, 52 remain disconnected from each other and may be pulled separately or concurrently to manipulate the drain member 14 relative to abscess cavity 20 and the first and second incisions 24, 26. Manipulating the drain member 14 may help remove fluids 28 from the abscess cavity 20.

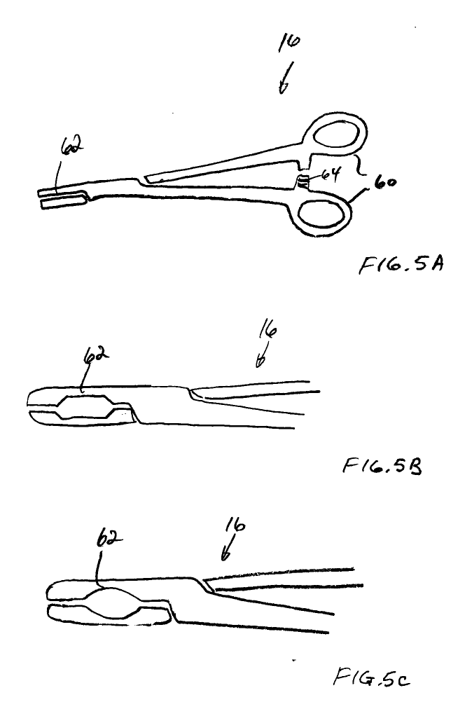

Referring now to FIGS. 2C and 5A-5C, a grasping member 16 may be used to handle or manipulate the puncture member 12. The grasping member 16 may grasp any portion of the puncture member 12 such as, for example, the proximal end portion 32 as shown in FIG. 2C. The operator may use the grasping member 16 to direct the puncture member 12 through the abscess, for example, to create the first and second incisions 24, 26. The grasping member 16 may also be directed through at least one of the first and second incisions 24, 26 during or after advancing puncture member 12 into and out of abscess cavity 20 to explore the abscess cavity 20 and break up pockets or loculations in abscess cavity 20.

Grasping member 16 may be a standard needle driver, a hemostat, or any other grasping device capable of grasping the puncture member 12 or drain member 14. FIG. 5A shows a standard hemostat having handles 60 and a grasping portion 62. The grasping portion 62 of FIG. 5A has a relatively planar or linear construction. FIGS. 5B and 5C show grasping portion 62 that have a preformed shape. The preformed shape may match an outer profile of the puncture member 12. The matching shape of the grasping portion 62 may provide improved contact between grasping member 16 and the puncture member 12. The grasping member 16 may include a locking feature 64 as shown in FIG. 5A to maintain a secure connection between the grasping member 16 and the puncture member 12.

The abscess draining system 10 described herein may include only the puncture member 12 and drain member 14. In other arrangements, the abscess draining system may additionally include the grasping member 16. A single drain member is positioned extending through the first and second incisions 24, 26 in the abscess cavity 20 according to the illustrations of FIGS. 1A-1E. Other embodiments may include positioning multiple drain members extending through the abscess cavity 20 through a single pair of the first and second incisions 24, 26, or through multiple pairs of first and second incisions 24, 26.

Other steps for treating the abscess may be used in addition to placing the drain member 14 as shown with reference to the figures. For example, the additional steps of applying a warm compress, applying manual pressure to the tissue layer 22 after creating one of the first and second incisions 24, 26, and other steps may be used to drain the fluids 28 and promote healing within the abscess cavity 20. In at least one example, a medication is applied via the drain member 14 using, for example, a coating applied to the drain member 14. Alternatively, the drain member 14 may include a plurality of holes or cavities that carry a medication or other treatment material that is transferred into the abscess cavity 20 after placing the drain member 14 as shown in at least FIGS. 1D and 1E. In still further arrangements, the drain member 14 is hollow and includes a plurality of perforations that provide fluid flow into the hollow center, and the fluids 28 pass through the hollow center of the drain member 14 to a location outside of the abscess cavity 20.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may he suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.”

What is claimed is:

1. A method of treating a cutaneous abscess, comprising:
providing a puncture member having a drain member attached to a proximal end of thereof;
inserting the puncture member through a skin surface and into the abscess, the puncture member forming a first incision;
advancing the puncture member from the abscess and through the skin surface to form a second incision, the drain member extending through the second incision, the abscess, and the first incision;
detaching the drain member from the puncture member;
opening the first and second incisions with the drain member to promote drainage of fluids from the abscess.

2. The method of claim 1, further comprising using a hemostat to insert and advance the puncture member.

3. The method of claim 1, wherein detaching the drain member from the puncture member includes breaking off the drain member.

4. The method of claim 1, wherein detaching the drain member from the puncture member includes cutting the drain member with a cutting feature of the puncture member.

5. The method of claim 1, further comprising forming a loop with the drain member after detaching the drain member from the puncture member.

6. The method of claim 5, wherein forming the loop includes tying opposing ends of the drain member together to form a knot.

7. The method of claim 1, wherein the puncture member has a tapered construction and advancing the puncture member through the first and second incisions dilates the first and second incisions.

8. The method of claim 1, wherein opening the first and second incisions with the drain member includes pulling the drain member away from the abscess.

9. The method of claim 1, further comprising dilating the first and second incisions with the puncture member.

10. A method of draining an abscess, comprising:
providing a puncture member, a drain member, and a hemostat;
connecting the drain member to a proximal end of the puncture member;
grasping the puncture member with the hemostat;
inserting the puncture member into the abscess with the hemostat at a first location and exiting the puncture member with the hemostat from the abscess at a second location to position the drain member extending through the abscess;
detaching the drain member from the puncture member;
articulating the drain member to drain fluid from the abscess.

11. The method of claim 10, wherein the hemostat has a grasping portion having a shape that matches a shape of an outer surface of the puncture member.

12. The method of claim 10, wherein the puncture member forms a first incision at the first location and forms a second incision at the second location.

13. The method of claim 12, wherein articulating the drain member includes opening the first and second incisions.

14. The method of claim 10, wherein the puncture member comprises a cutting surface, and detaching the drain member includes contacting the drain member with the cutting surface.

15. The method of claim 10, further comprising connecting opposing ends of the drain member together outside of the abscess prior to articulating the drain member.

16. The method of claim 10, wherein articulating the drain member includes pulling the drain member away from the abscess to enlarge openings into the abscess formed by inserting the puncture member through the abscess.

17. An abscess draining device, comprising:
a puncture member having a distal end configured to advance into and out of an abscess to form spaced apart first and second incisions;
a drain member connected to a proximal end of the puncture member, wherein advancing the puncture member through the abscess positions the drain member extending through the first incision, the abscess, and the second incision, the drain member being disconnectable from the puncture member after being positioned extending through the first incision, the abscess, and the second incision.

18. The abscess draining device of claim 17, wherein the puncture member is contoured along its length.

19. The abscess draining device of claim 17, wherein the puncture member is tapered along its length.

20. The abscess draining device of claim 17, wherein the puncture member comprises a cutting surface configured to sever the drain member from the puncture member.

21. The abscess draining device of claim 17, wherein the puncture member comprises an increased cross-sectional size from the distal end to the proximal end.

22. The abscess draining device of claim 17, wherein the drain member has a tubular construction.

23. The abscess draining device of claim 17, wherein the drain member has opposed first and second ends that are tied together to form a loop structure after the drain member is positioned extending through the first incision, the abscess, and the second incision.

24. The abscess draining device of claim 17, wherein one end of the drain member is inserted into the proximal end of the puncture member.
